(19) United States
(12) Patent Application Publication
(10) Pub. No.: US 2008/0243089 A1
(43) Pub. Date: Oct. 2, 2008

(54) Color-coded slide clamp

(76) Inventor: Candace Keaton, Pasadena, MD (US)

Correspondence Address: Ober, Kaler, Grimes & Shriver, 120 East Baltimore Street, Baltimore, MD, 21202-1643, US

(21) Appl. No.: 12/077,957

(22) Filed: Mar. 21, 2008

Related U.S. Application Data
(60) Provisional application No. 60/919,382, filed on Mar. 22, 2007.

(51) Int. Cl. A61M 39/28; F16K 7/04; F16L 55/10; A61M 5/00

(52) U.S. Cl. 604/250; 251/4

(57) ABSTRACT

A color-coded slide clamp for controlling the flow of fluid or other substance through an I.V. delivery tube. Each slide clamp generally comprises two longitudinal sides connected at one point by a resilient transverse end. At an opposing end, the longitudinal sides include crimping surfaces, which are clamped around the flexible delivery tube to regulate the flow of fluid passing there through. The precise width of the longitudinal sides determines the rate of flow of the fluid that may pass through the delivery tube. The wider the sides are, the lower rate of fluid that may pass through the tube. The width of the sides is fixed at the time of manufacture to correspond to a single, steady flow rate. The flow rate of the clamps can be easily identified by a color coding scheme. Alternately, clamps can be color-coded for easy identification of the type of fluids passing through the tubes.

### CROSS-REFERENCE TO RELATED APPLICATION(S)

The present application derives priority from U.S. provisional application Ser. No. 60/919,382 filed Mar. 22, 2007.

### BACKGROUND OF THE INVENTION

1. Field of the Invention

The present invention relates generally to subcutaneous (IV) systems and equipment and, more particularly, to a colored (color-coded) slide clamp which is employed to seal the fluid passageway of flexible tubing and identify the substance or flow rate of the substance passing through the tubing.

2. Description of the Background

Subcutaneous or IV systems deliver fluid, medications, blood products and parenteral nutrition to patients. Most IV machines operate by gravity or by an infusion pump (a pump, usually peristaltic, used to control the flow through the IV tube) or by a syringe pump (a pump, usually motor driven using an actuator assembly pushing the plunger of a syringe thus infusing into a patient via a subcutaneous infusion set connected to the syringe). A conventional IV administration system includes an IV machine with injectable solution bag, opening to a drip chamber, connected by flexible tubing to a tubing clamp and on to a subcutaneous infusion set for administration to a patient. These subcutaneous infusion sets typically include a needle/catheter attached microbore tubing, and some means of anchoring the needle/catheter to the skin of the patient. The tubing clamp may be crimped onto a delivery tube to control flow rate of the substance flowing through the tubing to the patient. Oftentimes, it is desirable to control the flow rate of the fluid flowing through the tubing to the patient, depending on the patient's status and medication or blood level. Physicians often desire that medical fluids be delivered to a patient with precision. Adjusting the clamps manually does not allow for precise control over the flow rate and total amount of fluid delivered. Improper control over the flow of the fluid through the tube could lead to serious consequences such as free flow hazard, which is a situation that occurs when fluid is free to flow through the delivery tube without regulation.

Also, many times the patient may have medication as well as blood or plasma administered to him through separate flexible tubes and the nurse or other health care professional who is managing the patient may utilize labels to identify the different substances passing through the separate tubes, or the health care professional must spend valuable time analyzing the tubing and its connections to assess which medication flows through which intravenous access point. When nurses or other health care providers change shifts, they must communicate the fluids passing through tubes as well as their respective flow rates. This can be a time-consuming, cumbersome, and confusing process.

Several types of slide clamps have been proposed in the prior art, however none teach or suggest colored slide clamps which allow for easy identification of the substance or flow rate of the substance passing through a delivery tube. Thus there exists a need for a colored slide clamp, which can be easily utilized by health care professionals to quickly and uniformly maintain the flow rate or quickly identify the substance administered to the patient.

It would be much more advantageous to provide a colored clamp that simplifies communication regarding patient care and yet still maintains patient safety while delivering fluid or other medical substance to a patient intravenously.

### SUMMARY OF THE INVENTION

It is the primary object of the present invention to provide a colored slide clamp that simplifies communication regarding patient care and yet still maintains patient safety.

It is therefore an object of the present invention to provide a colored slide clamp that regulates the delivery of fluid through the delivery tube to a high degree of accuracy.

Another object of the present invention is to provide a colored slide clamp that indicates the flow rate of the fluid that circulates within a delivery tube.

It is another object to provide a colored slide clamp that allows for quick and easy identification of the fluid circulating within a delivery tube.

Yet another object is to provide a colored slide clamp, which adds color and helps brighten the mood of the patient in a drab hospital environment.

Another object of the present invention is to provide a colored slide clamp that possesses a simple and scalable design.

It is another object of the present invention to provide a colored slide clamp that is fabricated of lightweight materials providing an appropriate degree of flexibility, resiliency, durability, and longevity.

An additional object of the present invention is to provide a colored slide clamp that is inexpensive to manufacture and sell to provide for widespread use.

In accordance with the foregoing objects, the present invention provides a colored slide clamp, which controls the flow of fluid or other substance through an I.V. delivery tube. The slide clamp generally comprises two longitudinal sides connected at one point by a transverse end. At an opposing end, the longitudinal sides include crimping surfaces, which are clamped around the flexible delivery tube to regulate the flow of fluid passing there through. The precise width of the longitudinal sides determines the rate of flow of the fluid that may pass through the delivery tube. The wider the sides are, then the lower rate of fluid that may pass through the tube. The width of the sides is fixed at the time of manufacture to correspond to a single, steady flow rate. The flow rate of the clamps can be easily identified by a color coding scheme. Alternately, clamps can be color-coded for easy identification of the fluids passing through the tubes.

### BRIEF DESCRIPTION OF THE DRAWINGS

Other objects, features, and advantages of the present invention will become more apparent from the following detailed description of the preferred embodiment and certain modifications thereof when taken together with the accompanying drawing in which:

FIG. 1 is a perspective view of an array of colored slide clamps 1 each in use with a subcutaneous infusion set 3.

### DETAILED DESCRIPTION OF THE PREFERRED EMBODIMENTS

The present invention is a color-coded slide clamp 1 for controlling the flow of fluid or other substances through an IV delivery tubes 2. The slide clamp 1 seals the fluid passageway of flexible tubing 2, thus restricting the flow of fluid there through.

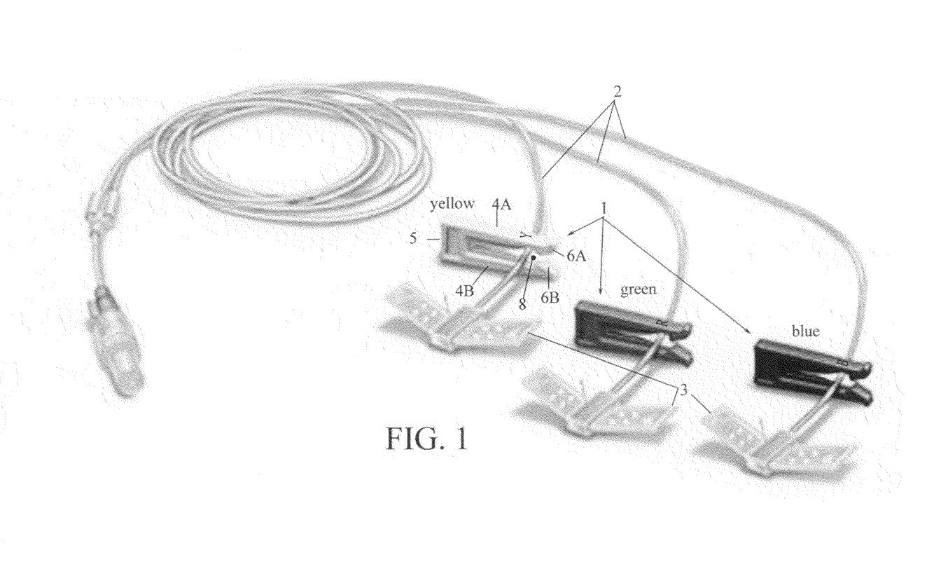

FIG. 1 is a perspective view of an array of colored slide clamps 1 according to the present invention, each in use with a subcutaneous infusion set 3. Each slide clamp 1 generally comprises two longitudinal sides 4A, 4B integrally connected at one end 5. At an opposing end, the longitudinal sides 4A, 4B include crimping prongs 6A, 6B with inward jaws defining a slot having a non-occluding section 8 leading outward to a pinch zone more proximate to the end 5, in which the slot is narrower than the occluding section. The tubing 2 can be selectively positioned within the non-occluding section 8 or moved into the occluding pinch zone. In this manner, the crimping prongs 6A, 6B of the clamp 1 may be clamped around the flexible delivery tube 2 to regulate the flow of fluid passing there through. Beyond the clamp 1, the tube 2 extends to a subcutaneous infusion set 3. The precise width of the longitudinal sides 4A, 4B determines the rate of flow of the fluid that circulates within the delivery tube 2. The wider the sides 4A, 4B are, the lower rate of fluid that may pass through the tube 2. The width of the sides 4A, 4B is fixed at the time of manufacture to correspond to a single, steady flow rate. Thus, the colored clamp 1 regulates the flow of fluid to a high predetermined degree of accuracy.

In accordance with the present invention the flow rate of the clamps 1 is easily identified by a color coding scheme. For example, red colored clamps correspond to 1.0 ml/hour, orange colored clamps correspond to 10.0 ml/hour, yellow colored clamps correspond to 50.0 ml/hour, green colored clamps correspond to 100.0 ml/hour, blue colored clamps correspond to 200.0 ml/hour, and violet colored clamps correspond to 500.0 ml/hour. Illustrated are a yellow colored clamp 1 (left) corresponding to 50.0 ml/hour, a green colored clamp (middle) corresponding to 100.0 ml/hour, and a blue colored clamp (right) corresponding to 200.0 ml/hour. The slide clamp 1 may be manufactured in a variety of colors each corresponding to a predetermined flow rate. One skilled in the art will understand that the aforementioned example is merely illustrative, rather than exhaustive of the colors and corresponding flow rates of the slide clamps 1. According to the patient status and medication level, the flow rate can be easily selected (and hence the colored clamps 1 can easily be identified and utilized). Alternately, clamps 1 can be color-coded for quick and easy identification of the fluids passing through the tubes 2. For example, the yellow colored clamp 1 (left) may indicate that blood or plasma is passing through the tube 2, the green colored clamp (middle) may indicate that a pharmaceutical is passing through the tube 2, and the blue colored clamp (right) may indicate glucose.

The colored clamps 1 may be easily manufactured by injection molding from colored plastic material dyed as desired. The clamps 1 are inexpensive to manufacture and sell to provide for widespread use. Additionally, the clamp 1 possesses a simple and scalable design, and is fabricated of lightweight materials providing an appropriate degree of flexibility, resiliency, durability, and longevity. Preferably, in addition to color codes, each color-coded clamp is embossed or molded with a corresponding label, such as the first initial of the corresponding color: “Y” for yellow, “R” for red, and “B” for blue (as illustrated), for technicians who may be color blind or otherwise visually impaired.

The colored slide clamp 1 simplifies communication regarding patient care through non-verbal symbols and enhances patient safety through the adoption of a standardized protocol. Additionally, the slide clamps 1 add color and help to brighten the mood of patients in a drab hospital environment.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

I claim:

1. In combination with an infusion system including a plurality of flexible fluid carrying tubes connected to infusion sets to deliver fluids to subcutaneous tissue, a plurality of color-coded slide clamps each comprising two longitudinal side members resiliently joined at one end, and extending to opposing jaws at another end, said jaws defining a crimping surface formed with a non-occluding recess leading to a pinch zone, whereby said crimping surfaces clamp around one of said flexible fluid carrying tubes to regulate a flow of fluid passing there through at a predetermined flow rate, and each of said plurality of color-coded slide clamps bearing a relatively distinguishable color corresponding to and providing a visual indication of the predetermined flow rate.

2. In combination with an infusion system including a plurality of flexible fluid carrying tubes connected to infusion sets to deliver particular fluids to subcutaneous tissue, a plurality of color-coded slide clamps each comprising two longitudinal side members resiliently joined at one end, and extending to opposing jaws at another end, said jaws defining a crimping surface formed with a non-occluding recess leading to a pinch zone, whereby said crimping surfaces clamp around one of said flexible fluid carrying tubes to regulate a flow of fluid passing there through, and each of said plurality of color-coded slide clamps bearing a relatively distinguishable color corresponding to a particular type of fluid in said flexible fluid carrying tube.
